(19) United States
(12) Patent Application Publication
(10) Pub. No.: US 2015/0356701 A1
(43) Pub. Date: Dec. 10, 2015

(54) MONITORING AND ADAPTING A PATIENT'S MEDICAL REGIMEN AND FACILITATING COMMUNICATION WITH A CAREGIVER

(71) Applicant: Play-it Health, Inc., Kansas City, KS (US)

(72) Inventors: Kimberly Lynn Gandy, Kansas City, KS (US); Piero Ferrante, Kansas City, MO (US)

(21) Appl. No.: 14/732,186

(22) Filed: Jun. 5, 2015

Related U.S. Application Data
(60) Provisional application No. 62/008,653, filed on Jun. 6, 2014.

(51) Int. Cl. G06Q 50/22; G06Q 10/10; A61B 19/00

(52) U.S. Cl. CPC G06Q 50/22; A61B 19/50; G06Q 10/10

(57) ABSTRACT

A user engagement system such as a medical regimen monitoring the adherence of a patient. The user engagement system customizes the medical regimen to meet the needs of the patient. The system includes a predictive engine for generating a set of recommended attributes for the regimen by analyzing a set of user information, an implementation engine for displaying the regimen to the user and monitoring adherences, and a responsive engine for adapting the regimen to improve an adherence level upon the patient falling below a threshold level by determining a set of revised attributes likely to improve adherence.

### RELATED APPLICATIONS

This non-provisional patent application claims priority benefit, with regard to all common subject matter, of earlier-filed U.S. Provisional Patent Application No. 62/008,653, filed on Jun. 6, 2014, and entitled “COMPUTER PROGRAM, METHOD, AND SYSTEM FOR MONITORING A PATIENT'S MEDICAL REGIMEN AND FACILITATING CONTACT WITH A CAREGIVER.” The identified earlier-filed provisional patent application is hereby incorporated by reference in its entirety into the present application.

### BACKGROUND

1. Field

Embodiments of the invention are broadly directed to providing a user with a regimen. More specifically, embodiments of the present invention are directed to creating, implementing, monitoring, and adapting the regimen, such as a medical regimen, for the patient.

2. Related Art

Patient engagement systems provide predetermined educational materials for patients, especially those that have a new disease, diagnosis, or injury. Typically, these systems provide only as much education and benefit as the patient is willing to use them. Systems of the prior art have only limited effectiveness for several reasons. First, they are “one size fits all” in that they are created generically and not with the specific patient in mind. Second, the systems of the prior art fail to monitor the adherence of the patient. Third, the systems of the prior art fail to facilitate communication between a caregiver and the user. Fourth, the systems of the prior art fail to analyze the communication between caregiver and patient. Finally, the systems of the prior art fail to adapt and improve the medical regimen over time to improve the adherence by the specific patient. Fifth, systems of the prior art fail to communicate engagement data to the continuum of caregivers in a platform in which they can cooperatively evaluate engagement data.

### SUMMARY

Embodiments of the invention solve the above-mentioned problems by providing a user engagement system that is created and/or organized for a specific user. The system analyzes known information about the user to select attributes that would appeal to or be appropriate for the user. The system monitors the adherence and the progress of the user in completing the regimen. If the user is not adhering, the system may prompt an interview between the user and a third party, such as a caregiver. The system analyzes the interaction between the user and the third party. Finally, embodiments of the invention analyze the reasons for non-adherence to determine reasons for the non-adherence and attributes for the regimen that may improve adherence.

Embodiments of the invention are directed to a user engagement system such as a medical regimen monitoring the adherence of a patient. The user engagement system customizes the medical regimen to meet the needs of the patient. The system includes a predictive engine for generating a set of recommended attributes for the regimen by analyzing a set of user information, an implementation engine for displaying the regimen to the user and monitoring adherences, and a responsive engine for adapting the regimen to improve an adherence level upon the patient falling below a threshold level by determining a set of revised attributes likely to improve adherence.

Embodiments of the invention are directed to a computerized method for providing a regimen to a user, the method comprising the following steps: receiving, from a predictive engine, a set of recommended attributes for the regimen; creating the regimen based upon the set of recommended attributes; presenting an educational component of the regimen to the user; presenting a reminder component of the regimen to the user; monitoring activities of the user to determine an adherence level of the user; initiating, upon the adherence level falling below a threshold, communication between the user and a third party; sending, to a responsive engine, a set of adherence information indicative of the activities of the user; and receiving, from the responsive engine, a set of revised attributes for the regimen.

Embodiments of the invention are directed to a system for improving adherence by a user to a regimen. The system includes a predictive engine for determining a set of recommended attributes for the regimen. The system includes an implementation engine for displaying the regimen to the user, which monitors an adherence level of the user. The system also includes a responsive engine for determining a set of revised attributes designed to improve the adherence level of the user, wherein the revised attributes are based at least in part on an analysis of a non-adherence pattern displayed by the user. The responsive engine accesses an attribute pool to determine attributes that are likely to improve the adherence level of the user.

Embodiments of the invention are directed to a non-transitory computer readable medium having a computer program thereon. The computer program instructs at least one processing element to perform the steps discussed herein.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

### BRIEF DESCRIPTION OF THE DRAWING FIGURES

Embodiments of the invention are described in detail below with reference to the attached drawing figures, wherein:

FIG. 1 is a flow diagram illustrating an exemplary embodiment of the invention, in which various components of a system for generating, monitoring, and adapting a regimen;

FIG. 2 is a flow diagram illustrating general steps as performed by a predictive engine;

FIG. 3 is a flow diagram illustrating general steps as performed by an implementation engine;

FIG. 4 is an exemplary depiction of a graphical user interface of a home page of the regimen;

FIG. 5 is an exemplary depiction of a graphical user interface of a game associated with the regimen, as depicted on a smart phone;

FIG. 6 is an exemplary depiction of a graphical user interface of a reminder associated with the regimen, as depicted on a smart watch;

FIG. 7 is an exemplary depiction of a graphical user interface of an initial reminder to contact a caregiver;

FIG. 8 is an exemplary depiction of a graphical user interface of a scheduling interface;

FIG. 9 is an exemplary depiction of a graphical user interface of a secondary reminder to contact the caregiver;

FIG. 10 is a flow diagram of the facilitation and monitoring of an interview between the user and the caregiver;

FIG. 11 is an exemplary depiction of a graphical user interface of an alert displayed to a caregiver of a non-adherent patient;

FIG. 12 is an exemplary depiction of a graphical user interface of an adherence score sheet as presented to the caregiver;

FIG. 13 is a flow diagram illustrating general steps as performed by a responsive engine; and

FIG. 14 is a system diagram of an embodiment of the invention depicting various computing devices and their components.

The drawing figures do not limit embodiments the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current technology can include a variety of combinations and/or integrations of the embodiments described herein.

The invention provides various embodiments of a computer program, a method, and a system for engaging a user. The engagement facilitates the relationship between the user and any number of third parties and assists the user in successfully completing a regimen. Embodiments of the invention are directed to a user engagement system such as a medical regimen for monitoring the adherence of a patient. The user engagement system customizes the medical regimen to meet the needs of the patient and adapts the regimen to improve adherence. The system includes a predictive engine for generating a set of recommended attributes for the regimen by analyzing a set of user information, an implementation engine for displaying the regimen to the user and monitoring adherences, and a responsive engine for adapting the regimen to improve an adherence level upon the patient falling below a threshold level by determining a set of revised attributes likely to improve adherence.

Most of the following detailed description is broadly directed to the medical field as an exemplary field of use for the invention. In the medical field, the user is often a patient, the third party is a caregiver, and the regimen is a medical regimen. It should be appreciated, however, that this is only an exemplary use that should not be construed as limiting on the invention. Therefore, throughout this description the terms “user” and “patient” are used interchangeably. Similarly, the terms “third party” and “caregiver” are used interchangeably. Further, “regimen” and “medical regimen” are used interchangeably. Other exemplary fields to which the invention may be suitable are discussed below.

A few terms relative to the exemplary medical field will be discussed for clarity. As used throughout, “patient” means a natural person who has been diagnosed with a disease, who has been injured or wounded, who is recovering from surgery or other operation, or who has other medical needs. The patient need not be under the active treatment of a physician or nurse. By way of example, a patient could be recently diagnosed with diabetes, an athlete recovering from a torn ACL, a person recovering from heart by-pass surgery, a child dealing with brain cancer, an elderly person who needs help remembering to take his prescribed medications or beginning to show signs of dementia, an individual following a fitness/activity protocol, or an individual with a mental health issue. A patient could also be a person at risk for a disease, injury, or surgery for whom a health regimen has been recommended. For example, this could include military personnel at high risk of mental health issues secondary to events experienced in military service.

As used throughout, “caregiver” means any person performing, in at least some capacity, the function of assisting or aiding the patient with their medical regimen. The caregiver may have some amount of medical, nursing, chiropractic, physical therapy, or related training. In some embodiments, the caregiver may, from time to time, see the patient in person for medical, nursing, chiropractic, or physical therapy sessions. In other embodiments, the caregiver never sees the patient in person but only assists the patient via the embodiments of the invention. In some embodiments of the invention, caregivers may be avatars with voice morphed by human beings or with a computer generated voice responsive system, for example the Carnegie Mellon System.

As used throughout, “medical regimen” means any type of on-going treatment, monitoring, or education. The medical regimen may be regarding medical, nursing, chiropractic, physical therapy, or other related fields. The medical regimen may have hourly, daily, weekly, and/or monthly requirements associated with it. The medical regimen may alternatively or in addition have hourly, daily, weekly, and/or monthly suggestions or recommendations associated with it. A patient may also have multiple medical regimens that cover either a single or multiple diseases or conditions. The medical regimen can also change over time. In some embodiments, the medical regimen has been specifically prescribed or designed for a specific patient. In other embodiments, the medical regimen is generic and could apply to a number of different patients. Examples of medical regimens include monitoring blood sugar for a diabetic patient, monitoring activity level for an elderly patient, providing physical therapy exercises to a patient recovering from an injury, and providing educational materials to a patient recently diagnosed with sickle cell anemia. A medical regimen could also include treatment, monitoring, education, or any combination thereof.

Education is another exemplary field of use for the invention. In these embodiments, the user is a student, the third party is a teacher, and the regimen is an educational curriculum. Yet another exemplary field for the invention may be the field of employment. In these embodiments, the user is an employee, the third party is a supervisor, and the regimen is a work-related training program or list of tasks to be performed. Yet further embodiments of the invention are directed to law enforcement. In these embodiments, the user is a parolee or a person on probation, the third party is a probation officer or a parole officer, and the regimen is the legal requirements for the completion of the parole or probation. A yet further exemplary field may be the field of exercise and nutrition. In these embodiments, the user is a client, the third party is a personal trainer and/or dietician, and the regimen is an exercise and/or diet plan.

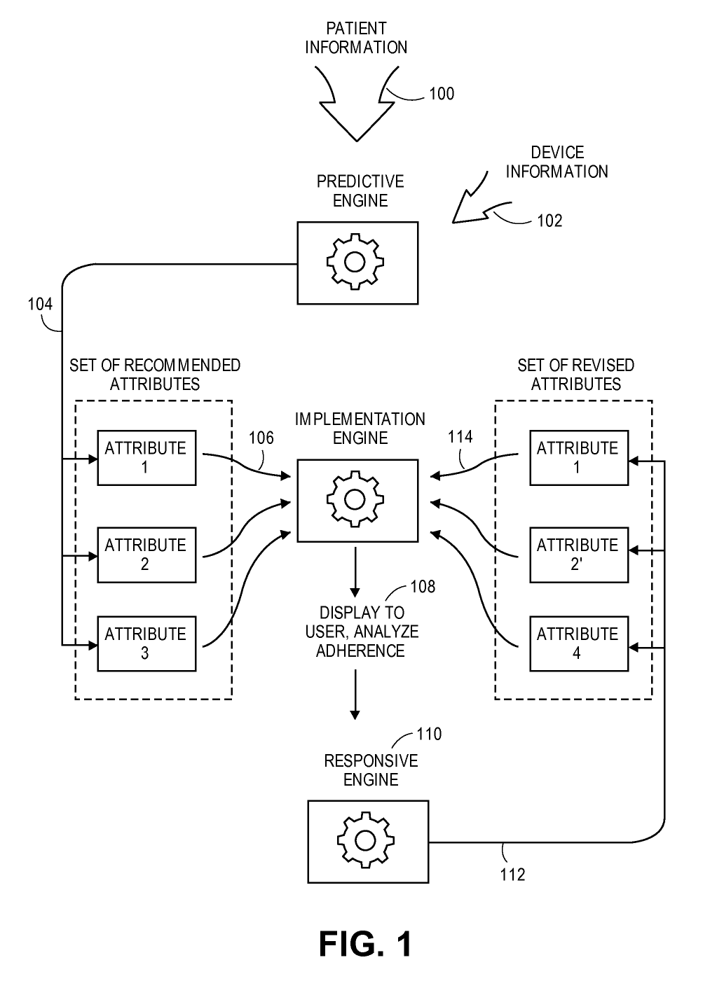

Turning to the figures, FIG. 1 is broadly directed to an exemplary embodiment of the invention. FIG. 1 broadly creates a regimen designed to meet the needs of a specific user, presents the regimen to the user, monitors the user's progress and adherence, and adapts the regimen to better meet the user's needs. In Step 100, the predictive engine receives user information, indicative of the type of user that will be utilizing the regimen. In Step 102, the predictive engine receives device information indicative of at least one device that the user will utilize to access the regimen. Based upon this information, in Step 104, the predictive engine draws on an attribute pool to determine a set of recommended attributes for the regimen. As illustrated, the set of recommended attributes includes “Attribute 1,” “Attribute 2,” and “Attribute 3.” The set of recommended attributes is then utilized by the implementation engine to display the regimen to the user in Step 106. The implementation engine also monitors and analyzes the user's adherence to the regimen. Adherence information is then sent to the responsive engine in Step 108. The responsive engine then analyzes the adherence information as well as any changes in the set of user information to create a set of revised attributes. As illustrated, the set of revised attributes includes “Attribute 1” (i.e. an unchanged attribute from the set of recommended attributes), “Attribute 2” (i.e. two prime, a revised or altered attribute from the set of recommended attributes), and “Attribute 4” (i.e. a new attribute not originally in the set of recommended attributes). The set of revised attributes is returned to the implementation engine in Step 114. The revised regimen is then again displayed to the user. Adherence information is again sent to the responsive engine for further revision of the attributes. The regimen therefore is repeatedly updated and revised to improve the adherence level of the user.

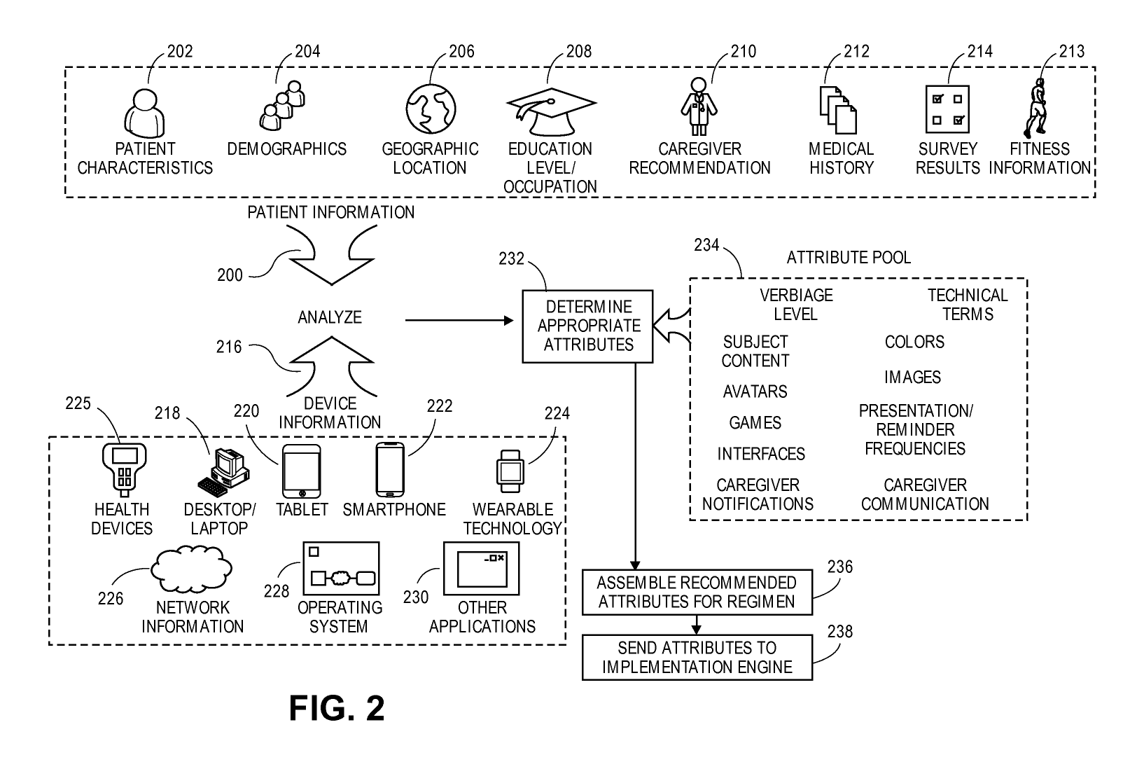

FIG. 2 is generally directed to steps performed by the predictive engine, including the creation and organization of the regimen based upon certain attributes. As discussed above, a common problem of patient engagement systems of the prior art is that they are not customized to the specific patient. As such, they are ineffective at meeting the specific needs and desires of the patient. It should be noted that FIG. 2 is expressed in terms of the medical field, but it is equally applicable to other fields.

The creation of the regimen begins in Step 200 with the analysis of available patient information. As described below, the patient information includes a portion, substantially all, or all available information about the patient such that the medical regimen can be tailored to the patient. In Step 202, the patient information includes patient characteristics, such as age, gender, and family composition. As an example, the patient characteristics can affect the incentives offered and avatars presented (discussed below). As another example, certain games may be appropriate to patients that are children, but would likely not appeal to adults.

In Step 204, the patient information includes demographics of the patient such as socio-economic class, race, marital status, parenthood, etc. For example, some educational modules may appeal more to parents than to non-parents. Demographics could also include a comparison of age to parenthood (i.e. a parent under 20 years of age, a non-parent over 30 years of age, etc.). Demographic information could also include an income level for the patient and/or the patient's household. The income level could be a numerical value or a range value.

In Step 206, the patient information includes a geographic location of the patient. The geographic location can affect the incentives offered and location-specific educational information. The geographic location could also include weather information for that geographic location. Such weather information could include information regarding the daily temperatures for the location, the amount of bright sun for the location, the amount of precipitation received, etc. The geographic location could also include information about the geographic location such as median income, median age, etc. The geographic location could also include information regarding nearby caregivers, hospitals, pharmacies, system administrators, and the like (along with the corresponding distances between such locations and the patients residence and/or work place).

In Step 208, the patient information includes an education level and occupation of the patient. As an example, the education level and occupation of the patient can affect the verbiage level, technical terms, and subject content of the medical regimen. Specifically, if the patient has an occupation in the medical field, more technical terms may be presented. If the patient has a low education level, few technical terms and a low verbiage level may be presented. If the patient is highly educated but not in the medical field, a high verbiage level with few technical terms may be presented. A related concept is proficiency in the language of the regimen, such as English. A person that is highly educated but has a low proficiency in English may require a low verbiage level for comprehension.

In Step 210, the patient information includes caregiver recommendations. The caregiver likely has an intimate knowledge of the patient as well as the patient's needs and desires. The caregiver is therefore invited to recommend certain attributes, discourage certain attributes, provide an analysis of the patient, etc. As an example, a caregiver may know that the patient has a high level of knowledge regarding the particular diagnosis (despite a low education level and non-medical occupation) and may therefore recommend a high verbiage level and many technical terms.

In Step 212, the patient information includes at least a portion of the medical history of the patient. As an example, the medical history of the patient can influence the technical terms, the subject content, and the caregiver notifications presented. The medical history can include a summary of the medical history, notably diagnoses or injuries in the medical history, etc. For example, a patient that is diagnosed with Type-2 diabetes may have some background knowledge if the patient was previously diagnosed with gestational diabetes. The medical regimen may draw analogies and contrasts between the two diagnoses. The medical history of the patient may also include information related to health-related actions taken (or not taken) by the patient. For example, the medical history could include the number of medication refills performed on time, the number of medication refills performed late, the number of medication refills not performed (along with whether or not the medication is considered critical for the patient), appointments made, appointments kept, appointments missed, etc.

In Step 214, the patient information includes survey results, qualitative information, and other patient-derived information. The survey may be presented by the system to the patient before the regimen, presented to the caregiver for asking to the patient, presented to the patient while the patient is seeing the caregiver, etc. The survey may include questions such as the desired topics of the patient, incentives that the patient might be interested in, games and avatars that might appeal to the patient, preferred method of contacting the patient, etc. The survey may also be presented periodically or on demand during the performance of the regimen.

In Step 215, the patient information includes activity information. Activity information could include daily step and exercise information. Exercise information could include the length, type, intensity, and regularity of the exercise. Exercise information could also include information such as heart rate information (such as resting heart rate, maximum heart rate, target heart rate, heart rate zones, heart rate during activity, etc.) Activity information could also include sleep information, such as the amount of sleep the patient is receiving per night on average, the number of nights in which the patient receives adequate sleep (e.g. 7-10 hours), the number of nights in which the patient receives inadequate sleep (e.g. fewer than 7 hours), the number of nights in which the patient receives excessive sleep (e.g. more than 10 hours), the number of nights in which the patient receives a less-than-normal amount of sleep (e.g. 20% less than normal), the number of naps taken, etc. As with other data sets discussed, the activity information may be obtained from an external program or device (such as an activity tracker application on a smart phone or a pedometer worn by the patient), or by a manual entry by the patient (such as the patient entering the heart rate detected by a heart rate monitor that does not communicate with the system).

Patient information could include other data sets. One data set that may be included in the patient information is information related to a purchase history for the patient. The purchase history information could include credit card usage frequencies, credit card fees charged, a percentage of charges above or below a certain normal range, purchases above a certain threshold (e.g. above $50, above $100, above 5% of monthly income), types of purchases (e.g. the percentage of purchases for clothing, food, etc.), amount of money spent on health-related expenses (such as medical insurance, copays, medical devices, etc.).

Another data set that may be included in the patient information is social media information. Social media information could include a number or frequency of posts, pictures posts, shares, likes, friends, new friends, un-friends, hashtags, tweets, favorites, retweets, categories, pages, etc. The social media information can also include the devices from which the user accesses the social media, the number and frequency of use, the average amount of time spent, etc. The system may access the social media information by providing a login screen to the user via the system. The system will then (with the permission of the user) log into the social media on behalf of the user and may remain logged in so as to track activity.

In Step 216, the system analyzes device information for the patient. In some instances, the device information is derived from the device that accesses the system, from the survey discussed in Step 214, an indication from the patient of the types of devices that they possess and are interested in being associated with the system, etc. Because some attributes, functions, and steps are more and less effective based upon the device platform, the device information may affect the set of recommended attributes.

In Step 218, the device information includes information indicative of a laptop or desktop computer. Laptop and desktop computers typically provide for the display of more information and faster processors for the computation of data and the presentation of media. In Step 220, the device information includes information indicative of a tablet computer. Some tablet computers (as well as laptop computers) may have periods without Internet connectivity due to the lack of a mobile broadband connection, which may affect the types of notifications and communication that they can facilitate.

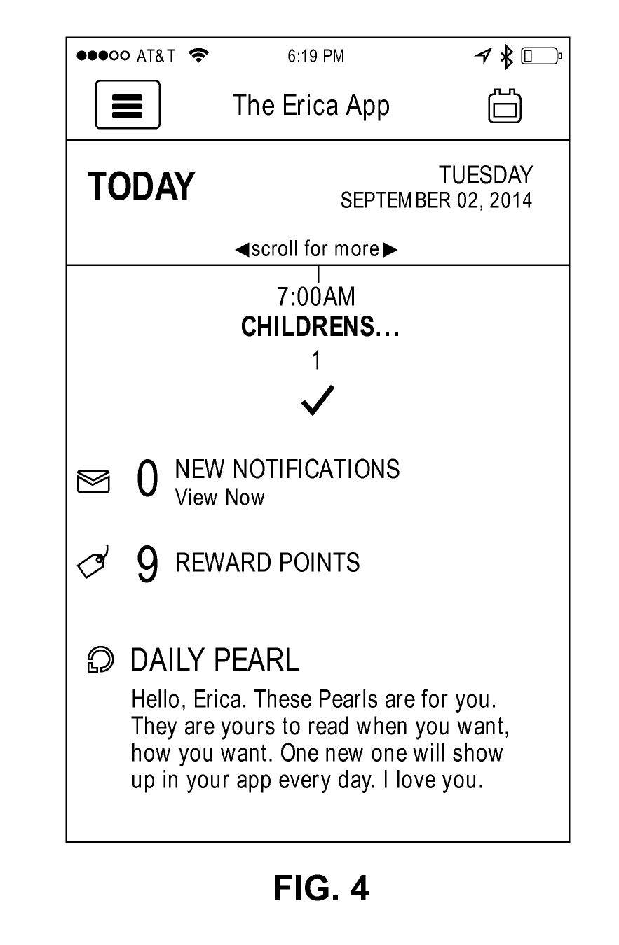

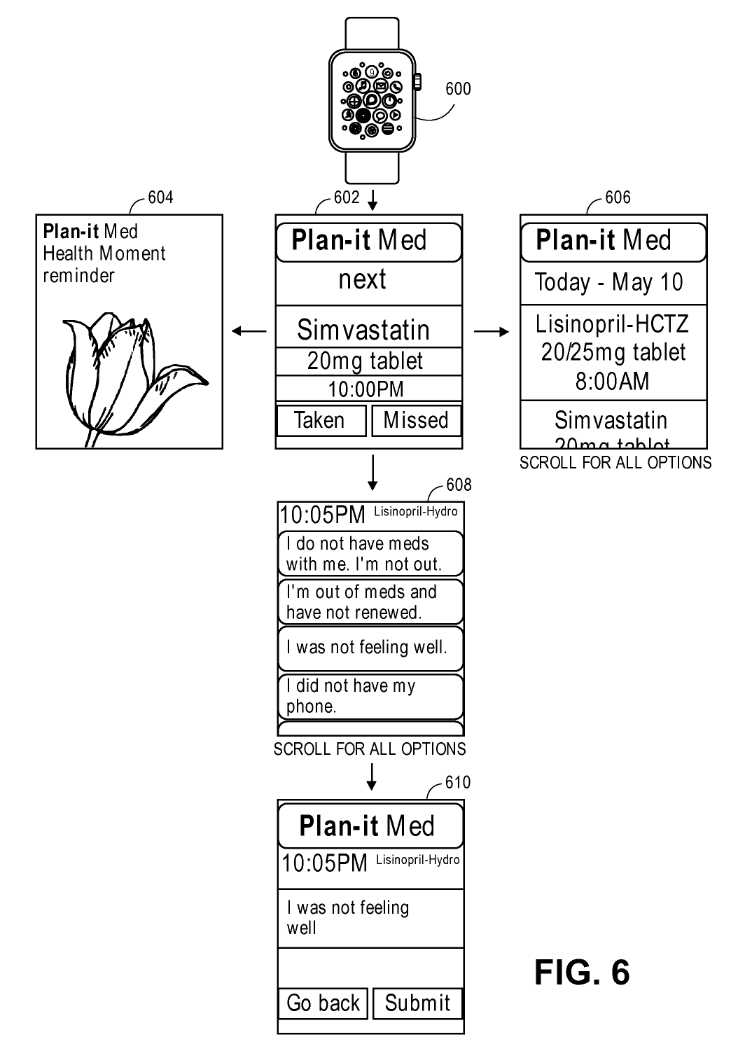

In Step 222, the device information includes information indicative of a smart phone. Examples of information formatted for a smart phone can be seen in FIGS. 4-5 and 7-9. The smart phone information (as well as other device information) can include usage statistics, such as screen-on time, active interaction time, call time, texts per day. In Step 224, the device information includes information indicative of a wearable technology such as a smart watch or the like. Wearable technology provides convenience for notifications, but limited computing power, screen size, and Internet access. Examples of information formatted for a smart watch can be seen in FIG. 6.

In Step 225, the device information includes information indicative of at least one medical device. Typically, the system does not run on the medical device, but instead interacts with the medical device to receive information. Medical devices can include any of a plethora of devices, such as blood glucose meters, blood pressure monitors, thermometers, activity trackers, pedometers, weight scales, mobility equipment, home testing kits, breath analyzers, drug testers, etc. In some embodiments, the medical device is a subcomponent of or otherwise associated with one of the above-discussed types of devices. In some embodiments, the medical device interacts with and shares information with the system. In some embodiments, the medical device does not interact with the system and the patient must transfer information between the medical device and the system. For example, the patient may enter reading information from the medical device. As another example, the patient may take a picture of the reading as displayed on the medical device. The reading may then be determined by optical character recognition (OCR) and determine that the test was in fact performed (so as to prevent the patient from fraudulently creating or altering test results). In some embodiments, the information can include whether the test or other action was performed, the time performed, the duration performed, the reading level (if applicable), associated thresholds for the reading level, etc.

In Step 226, the device information includes network information for the device or devices. The type of network, as well as the bandwidth of the network, may affect the type and size of attributes for the regimen. In Step 228, the device information includes information related to the operating system of the device or devices. Certain operating systems may enable certain versions of the software, certain features may be supported by some operating systems but not by others, etc.

In Step 230, the device information may include information indicative of other applications or computer programs present on the device or devices. The system may interact with, receive information from, or utilize the other applications in the determination of appropriate attributes and the presentation of the medical regimen. For example, if the device is a smart phone, the system may analyze the games installed on the phone to select a game for the regimen that would likely appeal to the patient.

In Step 232, the system determines appropriate attributes for the patient, based upon the patient information and the device information. In some embodiments, the system generates a patient profile based upon the analysis that includes information indicative of the type of patient and/or device. The system determines appropriate attributes by analyzing the available information to make a determination of the attributes and variations thereof that may appeal to the patient. Patients presented with a regimen that does not appeal to them are much more likely to fail in the adherence to the regimen. The predictive engine therefore increases the likelihood of adherence by customizing and adapting the regimen to a specific patient.

In performing Step 232, the system considers the possible attributes in Step 234. Exemplary attributes of an attribute pool are illustrated in FIG. 2. Each attribute may include various levels, versions, types, and contents. The verbiage level is the complexity of words, syntax, and sentence structure (and the like) utilized by the educational modules and other information presentation in the regimen. The verbiage level may be measured in a grade reading level, a generic ranking, etc. In embodiments of the invention, the verbiage level already exists for that educational module. For example, there may be three versions of the educational module on pancreatic cancer (a high verbiage level version, a standard verbiage level version, and a low verbiage level version). In other embodiments, the system creates or modifies existing verbiage to meet the analyzed patient information. Like many of the other attributes, attaining the proper verbiage level for the patient increases the patient's adherence by appealing to patient. Verbiage levels that are too low or too high for the patient are distracting and discouraging. Related to the verbiage level is the use of technical terms. As an example, the regimen may begin by using the technical terms (for patients with background knowledge), may slowly introduce the technical terms over time, or may avoid technical terms altogether.

The subject content is the topics and areas that the educational material covers. For example, the possible subject content for a diagnosis could include explanations of what the disease is, the causes of the disease, standard treatments for the disease, potential side effects of the treatments, the healing process, how the disease can affect normal life, background information on affected organs and bodily systems, etc. The system may select and prioritize the subject content based upon what the patient needs to know and what information may appeal to the patient.

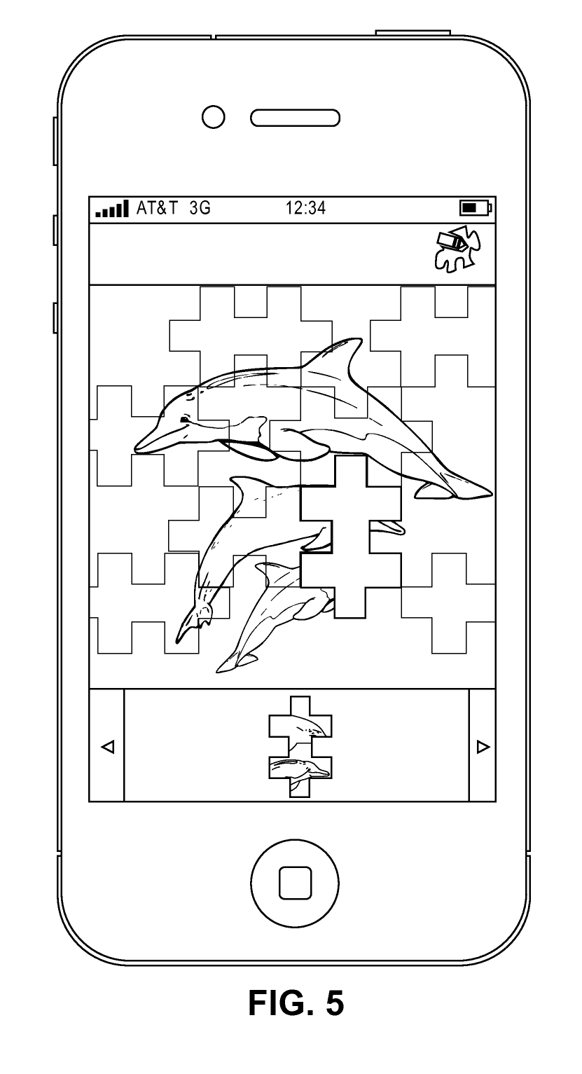

Other attributes in the attribute pool include colors, images, avatars, and interfaces that may be aesthetically pleasing to the patient. In some embodiments, the patient is invited to change these attributes. As shown in FIG. 4, the interface may include the layout of information presented. As shown in FIG. 5, the images may include background images for puzzles or other games. Still other attributes include the frequencies of the presentation of information and reminders. The frequencies should be often enough that the patient is learning and being reminded, but rare enough so that the patient does not become overwhelmed and fall behind. The system therefore estimates, based upon the above-discussed analysis, frequencies that will appeal to and be appropriate for the patient. For example, a patient that is a child may need more frequent reminders than a patient that is an adult. The type of reminder may also vary based upon the device information. For example, a reminder on a smart watch (as illustrated in FIG. 6) may differ from a reminder presented on a desktop computer. A related concept is the reminder verbiage escalation. Initial reminders may contain language that is friendly in character, but later reminders (following the dismissal or ignoring of earlier reminders) may include language that is more forceful and serious in character. The system may therefore select a reminder verbiage escalation rate that is appropriate for the patient. Some patients may be put off by reminder verbiage escalation while others may appreciate it. Yet a further attribute related to reminders may be an adaptive reminder that analyzes and responds to the patient's review and response to a reminder or a device to which the reminder is sent. For example, the system may analyze that the patient is slower to respond on weekends as compared to weekdays. In view of this analysis, the system may adaptively increase or decrease the time between reminders on the weekends. Alternatively, the system may analyze that the patient is quick to respond to reminders sent during a lunch break but slower to respond to reminders sent during the work hours. In view of this analysis, the system may adaptively send reminders closer to learned “break” times for the patient. Yet another adaptive attribute reminder process is analyzing that the patient reviewed the reminder on their smart watch. If the patient does not respond within a pre-set amount of time, the reminder may be sent again (based on a pre-set rule that the patient quickly reviewed and did not process the need to respond). However, if the patient reviewed the reminder on their tablet, more time for a response may be provided before another reminder is sent. As should be appreciated, various rules can be created and stored for implementation by the system. The system may also have access to the patient's personal and/or business calendar to note meetings/appointments/calls/vacations/out of office on the patient's calendar so that reminders are not sent during these hours.

Other attributes in the attribute pool may relate to caregiver notifications. These attributes may include the threshold initiations, type, information included, and frequency. Caregiver notifications can include positive reports, non-adherence reports, periodic reports, etc. Related attributes may include the initiation of caregiver communications, as discussed below. Reports may be delivered through online portals, e-mails, fax, or printed copy (e.g. traditional mail). These reports may be accessed by a single icon on a third party medical management system such as a health information exchange or electronic medical record.

In Step 236, the predictive engine assembles the above-discussed attributes that were selected into a set of recommended attributes. The set of recommended attributes may accompany the patient profile discussed above. In Step 238 the predictive engine sends the set of recommended attributes to the implementation engine for display to the user.

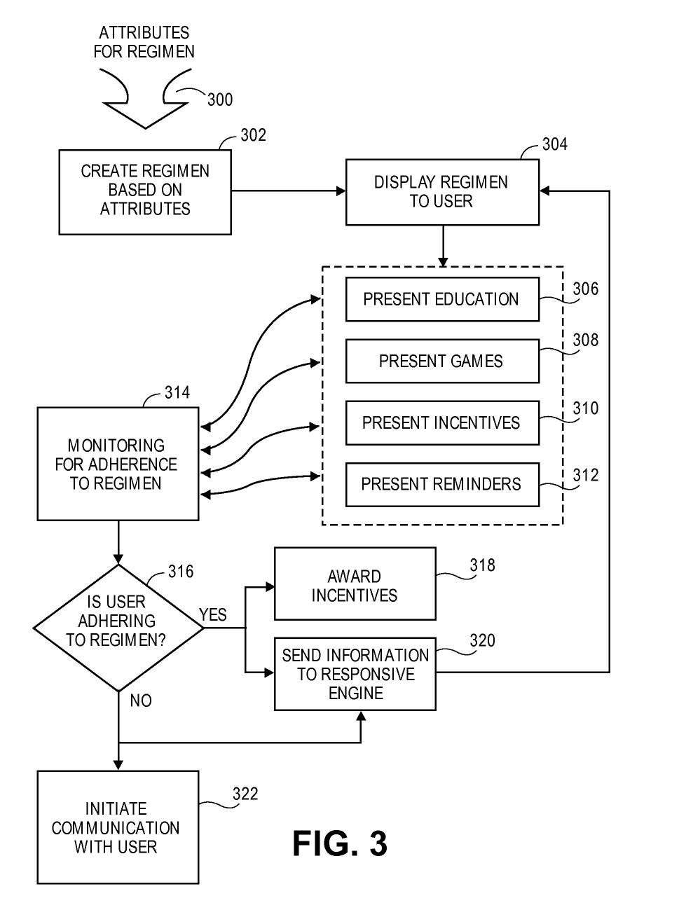

Turning to FIG. 3, attributes for the regimen are received, retrieved, or otherwise acquired by the implementation engine in Step 300. It should be noted, referring back to FIG. 1, that the attributes for the regimen can be the set of recommended attributes from the predictive engine, the set of revised attributes from the responsive engine, or even a further set of revised attributes from the responsive engine. The implementation engine, as illustrated in FIG. 3, displays and monitors the regimen regardless of origin.

In Step 302, the implementation engine creates or assembles the regimen based upon the attributes. In some embodiments, the implementation engine assembles the regimen from pre-existing attributes. For example, the implementation engine may retrieve educational material about diabetes from a data store, reminder information specific to the medications of the patient from the caregiver or a pharmacist, and overlay the information onto the recommended interface. In other embodiments, the implementation changes attributes in relation to an existing regimen. For example, the implementation engine may access a complete regimen for colon cancer, reduce the verbiage level to suit the patient, change the color scheme, and add a game that might appeal to the patient.

In Step 304, the implementation engine displays the regimen to the user. As discussed throughout, the regimen can be displayed on any combination of devices and interfaces (such as an internet website or smart phone application). The regimen can also be displayed over a length of time. For example, as shown at the bottom of FIG. 4, the regimen may include a daily educational module for delivery and display each day.

Typically, the regimen will include a home page, such as the one illustrated in FIG. 4. As illustrated, the home page may include the date, medication reminders (and an indication of their completeness), notification information, reward points information, and educational information (the “daily pearl” as illustrated). From the homepage, the patient can navigate to the various components and attributes of the regimen.

In Step 306, embodiments of the invention present educational modules to the patient. The education modules may be periodic, such as daily or weekly; a set total number of modules, to be completed by the patient; or as needed. An educational module is a set of data that contains information relevant to the patient's medical regimen and/or disease or condition. Educational modules may include any or all of simple text, stylized text, interactive learning games, videos, audio, questions, and answers to those questions. The system may inject questions in the educational modules to help optimize the data for the responsive engine. For example, a question such as “Which of the following rewards would you prefer” may be presented. Users will gain points (discussed below) for answering these question. Answers will be considered by the responsive engine when determining a set of revised attributes. In some embodiments, the question may be repeated in future educational modules to determine if answers change over time.

The educational modules may also provide electronic hyperlinks, web addresses, or other citation to external information. Educational modules can serve many purposes, such as providing the patient with information regarding a recent diagnosis, monitoring the patient's interaction with the system, refreshing the patient on topics previously discussed with the doctor, reminding the patient of the components of the regimen, and warning the patient of possible consequences of failing to follow the medical regimen. The educational modules can be optional or a necessary part of the medical regimen. In other embodiments, portions of the educational module are necessary and other portions are optional. The educational modules can be created specifically for the patient, selected from a set of existing educational modules, customized from the set of existing educational modules to fit the patient's specific needs, or provide generic medical information not specifically targeted to the patient.

In Step 308, the patient is presented with games, puzzles, and the like. Games and puzzles provide interest to the user, incentivize completion of the regimen, etc. An example of a puzzle is shown in FIG. 5. As illustrated the puzzle is presented to the user via the device. In some embodiments, the user may be presented a puzzle piece upon the completion of an education module, upon the answering of a question, etc. The user's curiosity at the completed puzzle provides motivation to the user to complete another module or component of the regimen. Some games have the potential for gamification, in which patients can evaluate accumulated points or progress in a game in the context of comparison to peers. Peer groups can be established by the user or by the system. For instance, system may establish a competition between Duke renal transplant and University of Kansas Renal Transplant for the highest adherence scores. There may also be rewards for encouraging peers to participate. For instance, children that encourage a buddy to engage in an adherence game could be rewarded with access to new games. Buddy or team mentality is therefore rewarded. If a patient has registered with a buddy or a team, and the buddy or team score improves, the group may rewarded as a whole.

In Step 310, the patient is presented with incentives for adherence. In embodiments of the invention, as the patient completes the educational modules, the patient is rewarded with points or credits. Points provide at least some of the incentive for the patient to complete the educational modules. The points can serve numerous purposes. The points can affect a level of adherence to the medical regimen, as discussed below. The points can also be redeemable for cash, gift certificates, and purchases at certain retail establishments. The points may be transferred to an external system for redemption, or may kept internally.

In Step 312, embodiments of the invention provide reminders to the patient. In embodiments, a reminder may prompt the patient to indicate their adherence to the daily requirements of a medical regimen. The reminder may indicate what the requirements are, or may be an indicator that the patient has failed to adhere to the requirements, or both.

An exemplary reminder is depicted in FIG. 6. As illustrated in Screen 600, the regimen is displayed on the smart watch via an icon. The reminder may be displayed as a notification on the device, such that it appears and alerts the user even if the user is not currently utilizing the associated application. In Screen 602, the user is presented with the reminder. As illustrated, the reminder may include the name of a medication to take, the dosage, and the recommended time. The user may select whether the medication was taken or missed. If the user indicates that he took the medication, the regimen may display a screen such as Screen 604 to provide other reminders such as to complete the educational module. The regimen may also display a schedule of all medications and other requirements for the day, such as illustrated in Screen 606. If the user indicates that the medication (or other requirement) was missed, the regimen may display a follow up question as to the reason. The regimen may display a list of possible reasons for the user to select, such as illustrated in Screen 608. Upon the selection of one of the options from Screen 608, Screen 610 confirms the selection and allows the user to submit to the system for later analysis. The reason for the missed requirement may considered in determining a set of revised attributes. For example, if the user is often missing requirements and selects “I do not have my meds with me. I'm not out.” (or a similar item), the revised attributes may include a reminder to take medications with the user when the user is leaving the house (such as in the morning, or if the user has an out-of-town trip on their calendar).

The reminder may be generated in response to a patient event falling below a pre-determined threshold level. The patient event may be any event associated with the patient's care, such as an activity or a biological value (e.g., certain blood pressure reading). The threshold level may then be a particular value associated with the patient event. For example, the patient event may be exercises the patient is to perform each day, and the threshold value is a pre-set number of the exercises. If the patient does not perform the exercises, as detected or determined by a sensor associated with the patient or as input by the patient, then a reminder may be generated. As another non-limiting example, the medical regimen includes a requirement that the patient is to monitor their blood sugar each day. If the patient's blood sugar is either not input or higher than the pre-determined threshold level, a reminder is generated.

The reminder may be associated with an image that is designed to change mental associations with a medical reminder. For instance, if the patient information has shown that the user loves flowers, the reminder may appear with an image of a beautiful flower, to create a fundamental change in the patient's perception and reaction to a reminder. The system may also select a certain image based upon other patient information.

In Step 314, embodiments of the invention receive, analyze, and track data indicative of adherence to the medical regimen. The system receives data though at least one method of input. The method of input can be through self-reporting by the patient, the input of external data by the patient or caregiver, the completion of educational modules by the patient, or through tracking of activity by a device. Self-reporting is the input of data by the patient to indicate what portions of the medical regimen they have completed. Self-reporting is the least reliable but simplest form of input. Self-reporting can input specific figures such as the number of exercises performed, or mark completed requirements such as taking medication, or both. The system can also receive data from an external device. Examples of this data from an external device can be the reading from a blood glucose meter or blood pressure sensor. The data from the external device may be input directly into the system by the external device, brought into the system by a third party data analytic system, brought in through a proprietary GUI interface, or may be manually input by the patient, caregiver, or other person. The system can also receive data by the tracking of activity by a device that is a part of the system. Examples of this include tracking, via a smartphone or tablet, a distance that the patient has walked, or the number of times that the patient has visited a local gymnasium.

External data may also be monitored and analyzed in connection with the regimen. Some of this data may require logging into or authorization of external resources such as websites and databases. Examples of this external data could include social media accounts and feeds, weather patterns, purchase histories, event histories, travel histories, motion detectors from a location associated with the user, activity tracker data (either in the same device as the regimen or an external device that interfaces), puerperal biosensor data, hormonal pattern data from biosensors, parallel data from other family members or peers, critical data about the medical condition or diagnosis, financial data, peer group purchasing trends, etc.

In some embodiments of the invention, the data that is received is associated with the patient's account so that it can be tracked and analyzed over time. In some embodiments of the invention, the data is also associated with the caregiver's account so the caregiver can monitor the adherence to the medical regimen. In other embodiments of the invention, the caregiver has access to all or a summary of the data associated with the patient's account.

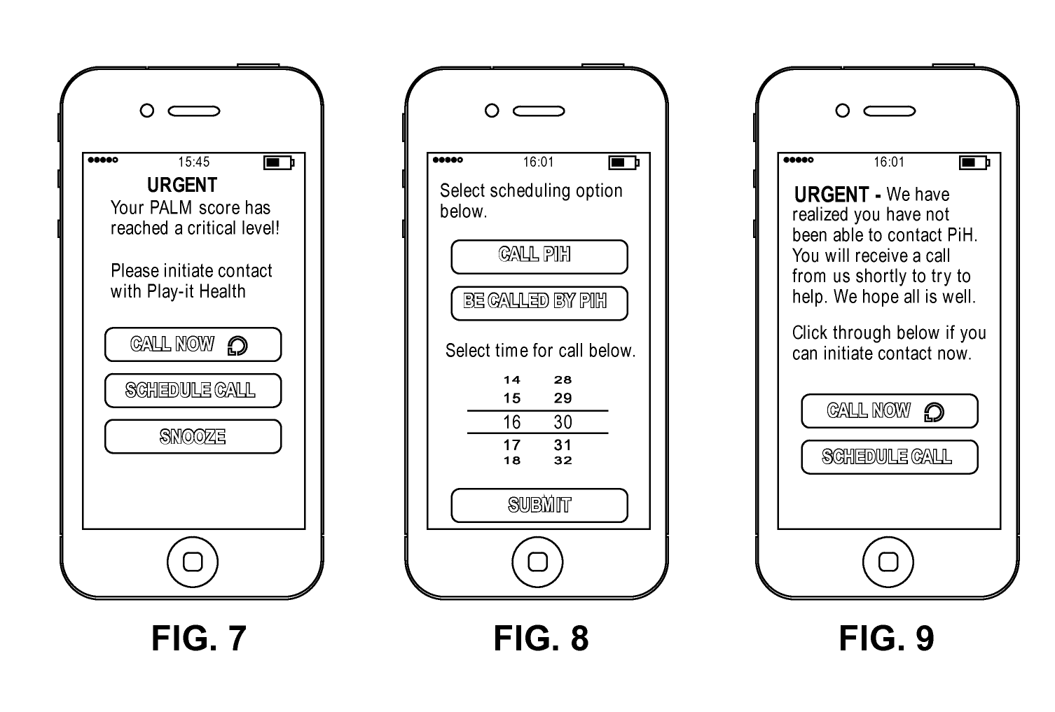

In Step 316, the system determines whether the patient is adhering to the medical regimen. The determination may be based upon a fixed threshold, a variable threshold, an average of adherence over time, or the like. The threshold may be expressed as a percentage, a number, a letter grade, color, ladder, or the like. There may also be multiple thresholds, a high threshold for a friendly reminder (such as illustrated in FIG. 7), a mid-level threshold for a more urgent reminder (such as illustrated in FIG. 9), and a low threshold for emergency situations (e.g. the caregiver may be contacted, emergency responders sent, etc.). The system may weigh certain types of adherence differently. For example, a patient that is failing to complete the educational modules but taking medication regularly may be adhering more than a patient who is failing to take medication but regularly completing the educational modules.

In some embodiments of the invention, the care provider monitors patients as a group to facilitate care management. When patients are incorporated into the system, default parameters are entered for critical levels of adherence for given elements (such as appointments, lab, educations, medications, physical therapy, education, etc.). A threshold is determined at which a patient should be placed on a “watch” list, and another threshold at which a patient should be placed on a “danger” list. These levels of adherence are initially defined by default thresholds within the system. The thresholds can be customized for a certain institution, diagnosis, or patient. For example, patients suffering from a certain chronic condition may be given higher thresholds for the “danger” list if failure to adhere can lead to more rapid health risks. When caregivers log into their portal (as discussed below), the classification of patients in these categories is displayed. Caregivers can also be notified, if desired, when these triggers are reached. Notification methods for such triggers can include text and e-mail.

If the system determines that the patient is adhering to the medical regimen, in Step 318 the system may present the patient with awards and points relative to the incentives that were discussed above in Step 310. In embodiments of the invention, the system provides points to the patient as the received data is indicative of adherence to the medical regimen. The number and type of points that will be awarded for each set of data indicative of adherence can be determined by the caregiver, a person administering the system, a person who created the educational modules or medical regimen, or the system. In embodiments, the points accumulate over time and do not expire. In other embodiments, the points expire periodically, after a certain period of time, or upon completion of the medical regimen. Patients who have two or more medical regimens may be able to pool their points between the medical regimens, or the points may be kept separately.

Embodiments of the invention reward adherence to the medical regimen. The rewards can take several forms, such as cash, gift certificates, purchases at retail establishments, credits toward services, coupons, points toward other incentive systems (such as airline miles), ability to make charitable donations, ability to donate points to a family member, or other objects of value. The rewards may be offered through the system or may be external. Points can be accumulated and cashed in as “gifts” to others. For example, a grandparent may accumulate their points to use for purchase of educational games for a grandchild. The system may present the grandparent with recommended choices for the grandchild, based upon any available information about the grandchild.

In Step 320, the system sends information to the responsive engine for further analysis. As can be seen in FIG. 3, Step 320 is performed if the user is complying and if the user is not complying. This is because the system attempts to improve adherence, even if the user is adhering adequately. This keeps the medical regimen from becoming stagnant and boring. However, upon the analysis of the responsive engine, the set of revised attributes may not change from the set of attributes currently being used.

If the system determines that the user is not adhering to the regimen, in Step 322 the system may initiate contact with the patient. In embodiments of the invention, some reminders are coupled with a request that the patient contact a third party, such as the caregiver, a system administrator, or other medical person. In these embodiments, other reminders will inform the patient of their failure to adhere to the medical regimen, or their failure to report their adherence. In yet further embodiments, the patient will be contacted immediately by the caregiver upon the patient event falling below the pre-determined threshold value or upon some other triggering event.

In some embodiments of the invention, the patient is presented an option to request contact with an adherence coach. In this embodiment, the adherence coach provides individualized coaching. In some embodiments of the invention, users may request a particular adherence coach or coaches. In other embodiments, the system selects a specific adherence coach based upon an analysis of the patient, the medical regimen, and the adherence level of the patient.

A potential adherence coach registers with the system or may be suggested through the caregiver. The system may present a pseudonym for the adherence coach to patients. Adherence coaches may offer these services voluntarily or may be compensated by the patient, the system, or the caregiver. For example, an adherence coach may be another patient with a similar medical diagnosis and the two patients act as adherence coaches for each other.

In embodiments of the invention, the adherence coaches have a rating based upon a number of factors, including a review given by patients who have worked with the adherence coach, a review given by other adherence coaches, a review given by a caregiver, and past success the adherence coach has achieved in improving adherence of the patients. In embodiments of the invention, adherence coaches receive points for the act of volunteering, a good review from patient, and good result in improving the adherence of patients. Similar to patients, the points may be used to receive rewards.

FIG. 7 provides an exemplary embodiment of an initial reminder to the patient informing them that the patient's level of adherence has reached a critical level, and requests that the patient contact the caregiver. As shown in FIG. 7, in embodiments of the invention, the GUI presents an initial reminder along with at least one option. The at least one option facilitate the patient contacting their caregiver. These options may include “Call Now,” “Schedule a Call,” and “Snooze.” Other options, or fewer options may be presented. In addition or in the alternative, other options may be shown or removed in a subsequent reminder should the patient fail to respond to the reminder (as illustrated in FIG. 9).

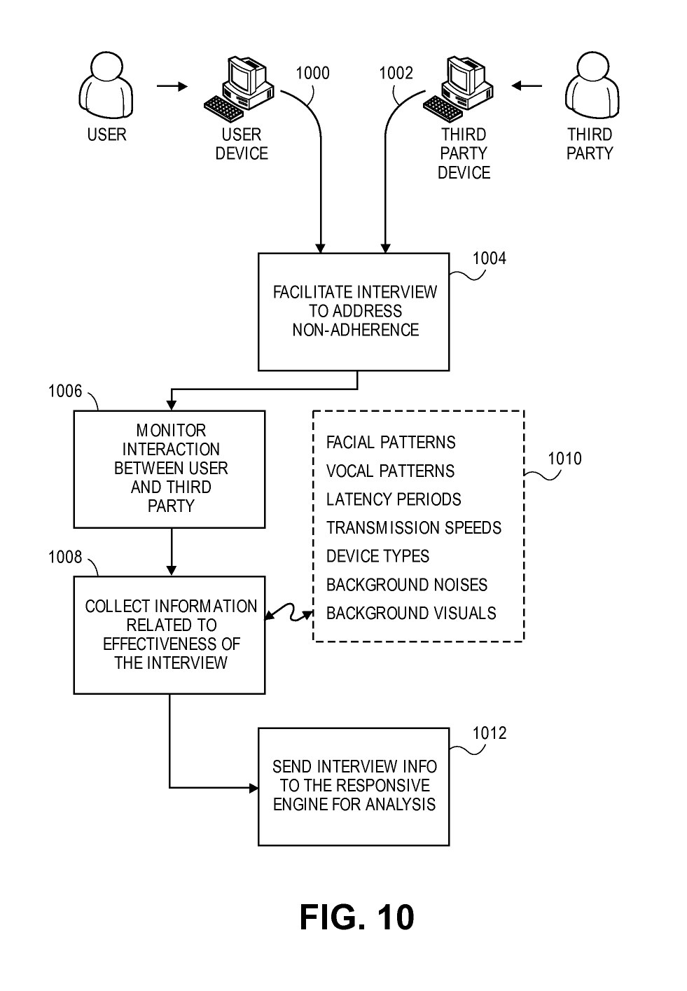

If the patient selects the “call now” option, embodiments of the invention facilitate communication between the caregiver and the patient, as discussed below in FIG. 10. This communication may take several forms, such as a traditional phone call, text message, or internet-based video chat function. The internet-based video chat function can be internal to the system, or link to an external system. Examples of these external systems include TELEMED, SKYPE, FACEBOOK MESSENGER, and GOOGLE HANGOUTS. In embodiments of the invention, the system facilitates communication by opening a new window or display within the GUI. In other embodiments of the invention, the system opens an external program or application through which the communication will take place. The communication is then analyzed as discussed below in FIG. 10.

Embodiments of the invention provide a snooze function with the reminder. The snooze function will present another similar reminder to the patient after a certain amount of time, or will present a reminder of the initial reminder. Upon the user selecting the snooze function, some embodiments of the invention present a snooze submenu providing snooze option. The patient may select the amount of time in which the patient would like to receive a subsequent reminder, such as “5 Minutes,” “10 Minutes,” and “15 Minutes.” In other embodiments of the invention, more or fewer options may be presented by the GUI, and in different intervals of time.

In other embodiments of the invention, the GUI does not present a snooze submenu and instead will execute a snooze function of a pre-determined amount. In still other embodiments of the invention, the patient may set their preferred snooze amount in an account settings presentation of the GUI. In embodiments of the invention, subsequent reminders may not provide a snooze option, or the snooze submenu may present only options of a relatively short period of time.

FIG. 8 provides an exemplary embodiment of the GUI presenting a scheduling submenu. In embodiments of the invention, the GUI presents the submenu upon the patient selecting the “Schedule a call” option. As illustrated, a GUI may present the patient with an option as to whether the patient would like to place or receive the call at the scheduled time. In other embodiments, the patient may not be presented with such an option. In still further embodiments, the patient may select their preferred method of contact in an account setting. In addition, the GUI may present the patient a representation indicative of times which the patient may place or receive the call. In embodiments, the system will verify what times are available to conduct the phone call and only display times which are acceptable. In other embodiments, the GUI presents all possible times, as shown below. In still further embodiments, the GUI presents the user the available times to the patient. For example, the available times may be listed as every single minute, five minutes, ten minutes, or half hour. In other embodiments, the GUI also presents the choice of a date for the patient to select. In still other embodiments, the choice of date is limited to the next two or three days. In yet further embodiments of the invention, the choice of a date is only displayed late in an afternoon or after business hours, for example.

Upon the selection of one or more options in the scheduling submenu, the system will schedule the call with the caregiver. This can be done in a number of ways, such as by sending an automated message to the caregiver's account, sending an automated message to the caregiver via e-mail or text message, placing a calendar event on an electronic calendar of the caregiver, or other method of communication.

In another embodiment, a set of parameters is listed in a database to facilitate the calling. The set of parameters may include information such as good times to call, preferred dates or days of the week, blackout dates and times, and calling preferences. Calling preferences could include a preference for a telephone call, text message, or internet-based video chat. When the designated date and/or time arrives, the system facilitates the communication between the patient and the caregiver. The system may also provide reminders, warnings, additional reminders, or confirmations before actually facilitating the communication.

FIG. 9 provides an exemplary embodiment of a presentation by the GUI of a subsequent reminder. In embodiments of the invention, a subsequent reminder is a presentation to the patient following inaction or lack of compliance after an initial or other subsequent reminder. As shown in FIG. 9, the subsequent reminder looks similar to the initial reminder of FIG. 7, but may contain more information, more urgent wording, etc. In embodiments of the invention, the patient is not presented the option to snooze the subsequent reminder. In embodiments of the invention, the patient is not presented with the option to schedule a future call, but makes an immediate call or selected a limited number of snoozes. These embodiments may be appropriate if the user's failure to adhere is potentially dangerous to health.

Turning now to FIG. 10, embodiments of the invention are directed to a facilitated conversation (an “interview”) between a patient and a caregiver and an analysis thereof. The interview can be through a personal computer, a simple phone conversation, and communication through an avatar, etc. The interview may be instigated by non-adherence, periodic, initiated by the user, etc.

In Step 1000, the user (being the patient in the medical field) connects via the user device to the system. The connection may be made through the system, such as TELEMED (as discussed above) or through an external program or application, such as SKYPE, FACEBOOK MESSENGER, and GOOGLE HANGOUTS. In Step 1002, the third party (being the caregiver or adherence coach in the medical field) connects via a third party device to the system. In other embodiments, the interview may be in-person, and the interview is recorded such that the system can perform the analysis described below.

In Step 1004, the system facilitates the interview to address the non-adherence and other concerns and questions of the user. During the interview, the third party and the user interact to address the non-adherence. Typically, the third party attempts to determine the reasons for the non-adherence in a helpful and productive manner.

In Step 1006, the system monitors the interaction between the user and the third party. In Step 1008, the system collects information during the monitoring related to the effectiveness of the interview based upon certain data sets 1010. In some embodiments, the collection of information is performed substantially in real time (as discussed below). In other embodiments, a set of video data associated with the interview is sent to the responsive engine for analysis of the data sets 1010. In some embodiments, the analysis on the effectiveness is provided back to the third party substantially in real time such that the third party can change various aspects of their interaction with the user so as to be more effective in the interview.

In some embodiments, the data sets include facial patterns based upon facial recognition. The facial patterns may be indicative of the mood of the user, a pain level of the user, and/or the mood of the third party. In some embodiments, the data sets include vocal patterns that may be indicative of the mood or pain level, tone of voice, and persuasiveness. In some embodiments, the data sets include latency periods, i.e. the periods without active speaking by either party. Excessive latency periods can lead to an ineffective interview, but can also be indicative of a normal conversation flow. The system therefore analyzes the lengths, frequency, and consistency of the latency periods.

In some embodiments, the data sets include transmission speeds, video resolutions, interruptions, and other technical aspects of the interview. These can be distracting, cut an interview off before completion, and discourage future interviews. The system therefore analyzes these technical aspects when analyzing the effectiveness of the interview. Similarly, the data sets may include information on a type of device used by the patient and caregiver. The interview effectiveness can depend on the type of device utilized (and accordingly other types of devices and other modes of interviews may be recommended in the future).

In some embodiments, the data sets include background noises and background visuals. Background noises and background visuals, both on the side of the user and on the side of the third party, can be distracting for the other party. Embodiments of the invention therefore analyze the type, size, decibel level, motion, and other factors related to the background noise and/or background visual.

In some embodiments, the data sets include analysis of a transcript of the interview. The transcript may be generated and analyzed during or after the interview. The analysis attempts to determine patterns and structure to the unstructured interviews. The system may perform an initial analysis of the transcript by the vectorization of term frequencies and term frequency inverse document frequency. These vectorizations identify important words (or the root words thereof) and their frequency. The system may then determine the topics discussed by various methods such as latent semantic indexing, latent dirichlet allocation, and non-negative matrix factorization. The topics of conversation may be associated with user or the third party (e.g. to determine if topics are emphasized or downplayed by the party). The system may determine an indication of the sentiment expressed by the parties. The system may count positive, neutral, and negative terms and analyzes the ratio of the terms against each other. One example could be a ratio of positive terms to all terms. Certain terms and certain combinations of terms may be given greater weight.

Some embodiments of the invention attempt to improve the performance of future interviews (with the same or other users) by determining phrases and sentences that are particularly effective or ineffective. The system may analyze complete sentences or phrases and compare them to other comparable sentences or phrases in other historical interviews. The determination of comparable interviews may be by Euclidean distances, Pearson correlation coefficients, or the like. The system may then analyze interviews that are effective in improving adherence (via the subsequent analysis discussed below). The system produces cluster models in a hierarchy to determine certain phrases or sentences that are common among effective or ineffective calls. The system may also estimate the effectiveness of a certain call based upon its similarities and differences with other prior calls.

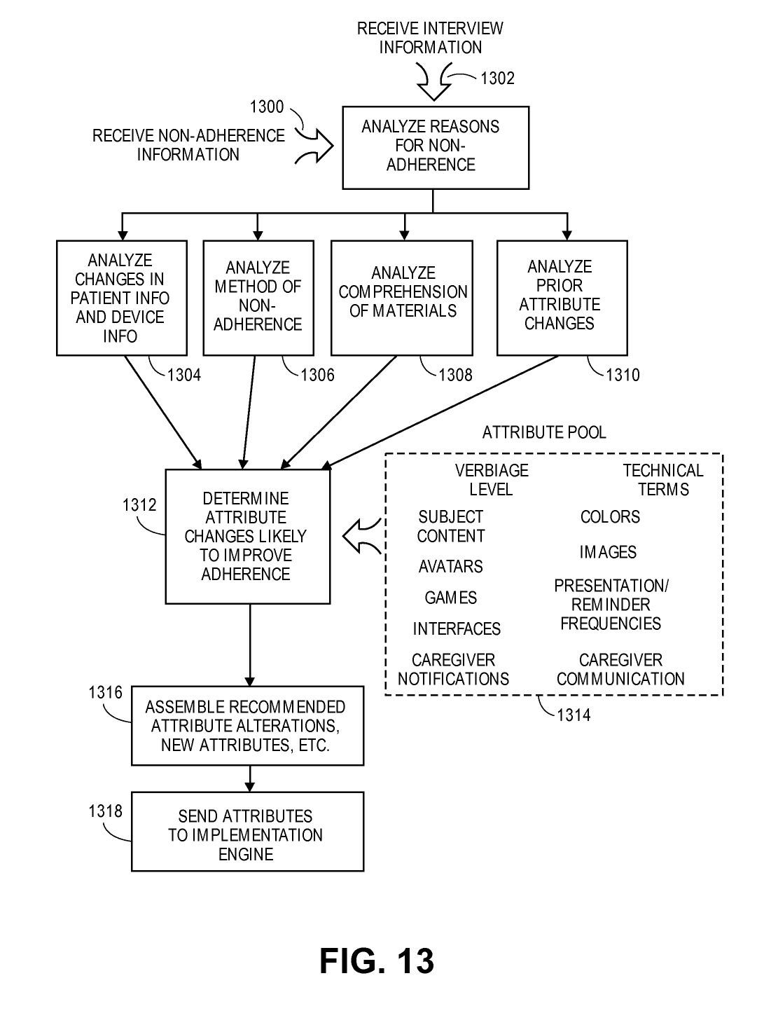

In Step 1012, the system sends interview information, including the above-discussed data sets, to the responsive engine for further analysis and comparison to other non-adherence information and the like. The analysis is further discussed below in FIG. 13. The purpose of the analysis is to determine a set of revised attributes based upon the topics discussed, a plan of action created, and the above-discussed data sets.

In embodiments of the invention, the patient is awarded points for making the communication. In these embodiments, the system verifies that a communication has taken place, and may record data such as length of the communication and the participants. In certain embodiments, the patient's non-adherence to the medical regimen is reset or voided after completion of the communication. In other embodiments, the caregiver provides input to the system as to whether the non-adherence should be reset or voided.

In embodiments of the invention, if the communication is not answered by the caregiver, the patient will be awarded points and presented an option to rescheduled the communication. In embodiments of the invention, the non-adherence is not reset or voided until person-to-person communication is made. In embodiments of the invention, if no response is received from the patient, a critical reminder responds periodically until a response has been received. The period of such critical reminders can be every hour, every two hours, every day, etc. In still other embodiments of the invention, a detection system detects through a mobile device or tablet if and when a communication is made. The detection system automatically reports the successful communication, so that points can be awarded and non-adherence reset.

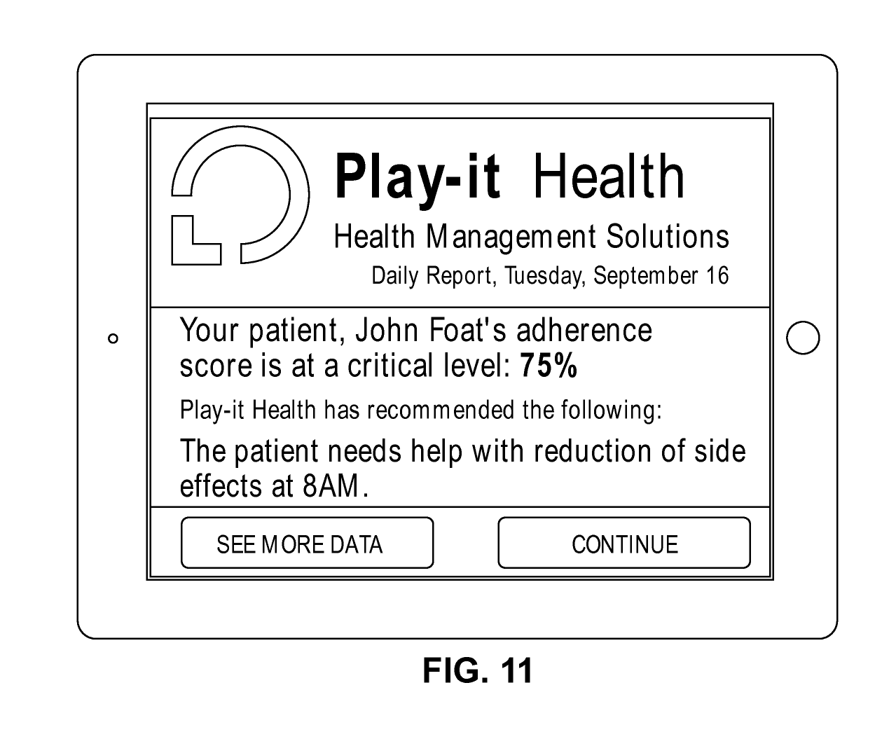

Embodiments of the invention are directed to the monitoring of the user by third parties. The third parties may be caregivers, adherence coaches, teachers, administrators of the system, etc. FIG. 11 depicts an exemplary alert that may be sent to a caregiver about the lack of adherence being displayed by their patient. The alert may provide information to the third party such as the adherence level, the name of the user, and recommended attribute changes from the responsive engine (discussed below). The alert may also make requests of the caregiver. In the exemplary alert of FIG. 11, the patient needs help reducing side effects. This information allows the caregiver to know the severity of the alert and develop a plan of action prior to contacting the patient (if necessary).

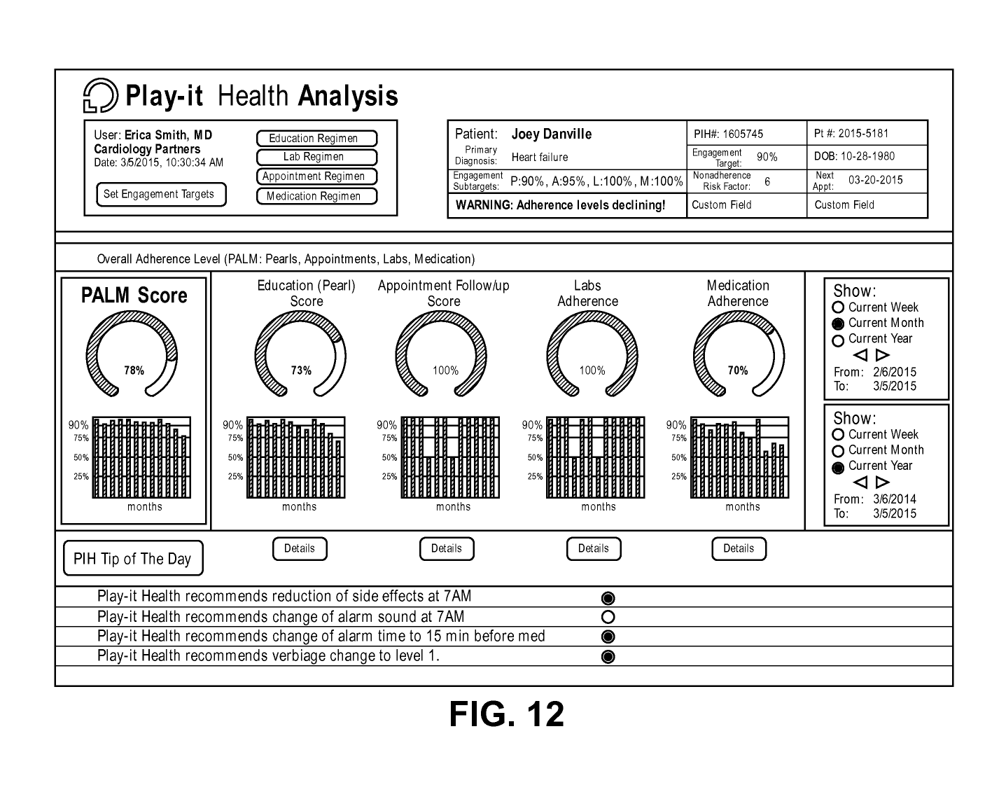

FIG. 12 depicts an exemplary patient score sheet. The score sheet contains additional information on the adherence of the patient. As an example, the caregiver or other third party may access the score sheet by selecting the “See more data” as illustrated on FIG. 11. The score sheet may break down the adherence into categories, such as educational adherence, appointment follow up adherence, lab adherence, medication adherence, and the like. As illustrated, the score sheet displays both current and past trends of adherence for each category. The score sheet may also display, or present an electronic link to, attribute information for the regimen. The score sheet may also display recommended attributes from the revised set of attributes and allow the caregiver to select and approve them. In some embodiments, the steps of the responsive engine (as illustrated in FIG. 13) are performed upon the caregiver requesting the score sheet, such that these recommended attributes are up-to-date. In other embodiments, the score sheet is presented to the caregiver following the steps of the responsive engine.

Some embodiments of the invention are directed to communication between various caregivers for the patient. For example, via a screen similar to FIG. 12, the caregiver may be invited to share information with other caregivers. Many patients are the patient or client of several caregivers. The medical regimen may therefore include requirements from multiple different caregivers. These requirements may be amalgamated or otherwise added together, but there may be unnecessary requirements, duplicative requirements, incompatible requirements, etc. For example, if a new caregiver is providing new requirements for an existing regimen, embodiments of the invention provide a means for the new caregiver to communicate with existing caregivers to verify compatibility and the like. Similarly, one caregiver can report the results of an in-office consultation or remote interview to other caregivers. This allows caregivers that are specialists or the like to be made aware of concerns or questions related to that specialty. This also helps all caregivers monitor progress of the patient and communicate amongst themselves to discuss strategies and attributes that may be effective.

Some embodiments also facilitate communication between the user and the caregiver or between the user and another external third party. For example, the user may send messages to their caregiver. As another example, the user may also inform the caregiver that they cannot make an appointment due to lack of transportation. The system may then authorize and contact a transportation company (such as a taxi company or the like) and may provide payment to the transportation company (the payment may come from the caregiver, the system, an insurance company, etc.).

In some embodiments of the invention, the system provides for secure messaging between caregivers and patients. Messages from the caregiver may appear as a notification, such as illustrated in FIG. 4. Examples of messages may include feedback and reminders from an appointment, notification that an insurance reimbursement has been obtained (i.e. prior authorization), and when appointments have been scheduled. The system may also allow for a patient response such as an evaluation of the appointment experience, follow-up questions, and the like. Information from these interactions will also be added into the information for analysis by the responsive engine.

In some embodiments of the invention, the system provides for secure messaging between a patient and a pharmacist. The system may provide an interface with a pharmacy that allows a patient to shop for the lowest price for a given medication or medical device in a certain geographic area. This interface can be accessed through the home screen or can be triggered when a patient indicates in the system that they did not take the mediation for financial reasons or needs to refill a certain medication. In some embodiments of the invention, the system also provides reminders to the patient and/or the pharmacy of the need for a refill. For example, the patient or pharmacist may input the number of pills provided to the patient, and the system will track as the patient takes each pill. The system can therefore know when the patient should refill the prescription and provide appropriate reminders.

In some embodiments of the invention, the system provides rewards and points to caregivers. As such, a reward component may interface with the clinician reporting dashboard or other caregiver interface. Caregiver receives points for interfacing with the system (e.g. reviewing patient reports, approving attribute changes, sending messages). For example, clinicians receive points for: clicking into the engine, clicks through the engine, comments to other clinicians through the dashboard. Points can be redeemed with the same platform that patients use.

FIG. 13 depicts exemplary steps of a responsive engine. Generally, the responsive engine collects and analyzes information about the regimen, the user's adherence to the regimen, changes in the user's situation, and the like. The responsive engine uses this analysis to determine which, if any, attributes should change, be added, or be removed from the regimen. Because the regimen is customizable, non-adherence can be addressed in effective ways. In some embodiments, the steps of FIG. 13 are performed periodically, such as daily, weekly, or monthly. In some embodiments, the steps of FIG. 13 are performed following certain events, such as interviews, medication refills, etc. In some embodiments, the steps of FIG. 13 are performed at the request of the patient, the caregiver, or other third party. In some embodiments, the steps of FIG. 13 are performed upon the patent falling below a threshold adherence level (the threshold level may also change over time). Any or all of the above-mentioned scenarios may be used to trigger the steps of FIG. 13. For example, the steps of FIG. 13 may be performed once a month unless there is an intervening fall in adherence or other triggering event.

In Step 1300, the responsive engine receives, retrieves, or otherwise acquires non-adherence information. As discussed above, the non-adherence information can include information gathered during the monitoring in Step 314 above, changes in patient information from Step 200, changes in device information from Step 216, information collected during the interview in Step 1008, etc.

In Step 1302, the responsive engine analyzes the available information to determine potential reasons for non-adherence. For example, if the patient rarely logs into the system, the reason for non-adherence may be a failure of the system to provide reminders that reach the patient. As another example, if the patient often answers educational questions incorrectly, the verbiage level or technical terms level of the educational material may be too high for the patient, and the patient may be failing to adhere out of frustration. A more detailed explanation of this analysis is given in Steps 1304-1310 below.

In Step 1304, the responsive engine analyzes changes in the patient information and device information since the predictive engine generated the set of recommended attributes. For example, a change in caregiver recommendations may be received following an interview or appointment with the caregiver. As another example, the patient may receive a new medical diagnosis that would change the medical history of the patient. Similarly, the patient may complete a new survey, or change answers on the already-completed survey reflecting changes in the desires of the patient. As yet a further example, the patient may have purchased a smart watch and desire a medical regimen more adapted to the smart watch.

In Step 1306, the responsive engine analyzes a pattern of non-adherence of the patient. Data indicative of a pattern of non-adherence includes statistics related to adherence scores, rewards acquired, rewards utilized, educational modules utilized, games utilized, reminders viewed, interviews conducted, caregiver appointments, inspirational/religious quotes utilized, etc. For example, the pattern of non-adherence may be a failing of the patient to report their adherence (i.e. the patient is adhering to the regimen but not reporting as such). As another example, the patient begins each educational module but does not complete the module. This method of non-adherence may be indicative of a patient that finds the educational modules too lengthy or too difficult to understand. The method of non-adherence is typically analyzed by looking at the behavior of the patient over time.

In Step 1308, the responsive engine analyzes the patient's comprehension of the materials presented. Comprehension may be demonstrated through the answering of questions related to the educational modules, the questions asked by the patient in the interview (and whether relevant information on the topic had already been presented in the educational modules), information provided by the patient as to desired topics to cover, etc.

In Step 1310, the responsive engine analyzes prior attribute changes recommended by previous iterations of the responsive engine. As best illustrated in FIG. 1, in embodiments of the invention the responsive engine and implementation engine run in a loop. After the set of revised attributes is sent to the implementation engine in Step 114, the revised regimen is again displayed to the user in Step 108, and adherence information is again sent to the responsive engine in Step 110. In Step 112, the responsive engine then analyzes the non-adherence information again to determine the effectiveness of the revised attributes. In Step 1310, the responsive engine considers at least a portion of, substantially all, or all of the previously-utilized attributes. The responsive engine may also consider previous iterations of the adherence information. For example, the responsive engine may consider how the set of revised attributes changed the adherence level. This consideration gives additional insight into how the attributes affect the adherence by the user and allows the responsive engine to continuously improve. In some embodiments, the responsive engine compares the adherence information to sets of attributes that have been effective for other comparable patients in the past or concurrently. If the patient shares characteristics with another patient that was previously failing to adhere, the responsive engine may analyze how the non-adherence was overcome.

In Step 1312, based upon the analysis, the responsive engine determines attribute changes, additions, or deletions that are likely to improve adherence. In doing so, in Step 1314 the responsive engine may access the same or another similar attribute pool, as discussed above in Step 234. In some embodiments, the attribute pool accessed by the predictive engine is more limited in the variations available than the attribute pool accessed by the responsive engine. For example, there may be three verbiage levels available to the predictive engine and five verbiage levels available to the responsive engine. As such, the predictive engine would not be permitted to select the highest or the lowest verbiage level but instead keep the selection in the center three options. The responsive engine may also request, from an administrator, an attribute that does not currently exist. For example, if the patient is a young child that is failing to comprehend educational materials at the lowest verbiage level, the responsive engine may request that an even lower level be created.

While examples of attributes and their corresponding levels have been discussed throughout (especially in Step 234 above), a few additional examples of attributes will be briefly discussed. Attributes can also include the timing of reminders, the tone with reminders, the voice utilized with reminders, the verbiage of reminders, background images and colors for educational modules or other interfaces, avatars associated with the educational modules, other visuals associated with educational modules, voice utilized in educational modules, slang and technical terms utilized by of avatars, expansion or retraction of the topic breadth for the educational modules.

One consideration when determining attributes is the number and type of changes as a whole. A drastic change of many attributes may reduce adherence by making the regimen unfamiliar to the patient, or it may make the regimen fresh and interesting to the patient. Incremental changes may not be noticed by the user, which can also be good or bad depending on the user. The responsive engine may also consider trends in adherence when determining the number and type of changes. For example, trends downward (i.e. toward non-adherence) may be indicative that a greater change would be effective in increasing adherence. Non-adherent but stable may be indicative that a smaller change would be effective in increasing adherence. Non-adherent and trending upward (i.e. toward adherence) may be indicative that no changes or slight changes would be effective in increasing adherence.

In Step 1316, the responsive engine assembles the attributes likely to improve adherence into a set of revised attributes. As discussed above, some attributes may remain unchanged, some attributes may be changed (e.g. moved to another level), some attributes may be deleted, and some attributes may be added (e.g. adding a game component). In some embodiments, the set of revised attributes is a complete set of all attributes in the set. In other embodiments, the set of revised attributes is a list of the changes, additions, and deletions to the set of attributes currently being utilized by the implementation engine.

In Step 1318, the responsive engine sends the set of revised attributes to the implementation engine such that the revised regimen may be displayed to the user, as illustrated in FIG. 1. In some embodiments, the user or caregiver may be presented with an option to implement the revised attributes (the caregiver's option to implement the attributes is illustrated at the bottom of FIG. 12). In other embodiments, the revised regimen is implemented and the user is presented with a message indicating that the regimen has been revised (in some embodiments, along with a list of the changes, additions, and deletions). In yet other embodiments, the revised regimen is implemented without notifying the user of the changes. In some embodiments, the implementation engine decides which of the above-discussed implementation methods to use based on the number and type of changes as a whole (as discussed above). For example, if the revised regimen has no changes or only minimal changes, the implementation engine may implement the changes without notifying the patient. If the revised regimen is drastically changed, the implementation engine may present a message to the patient explaining the proposed changes, recommending implementation, and presenting the patient with an option to agree. If the user likes the current regimen, the option allows the user to give themselves a second chance at adherence before submitting to the revised regimen.

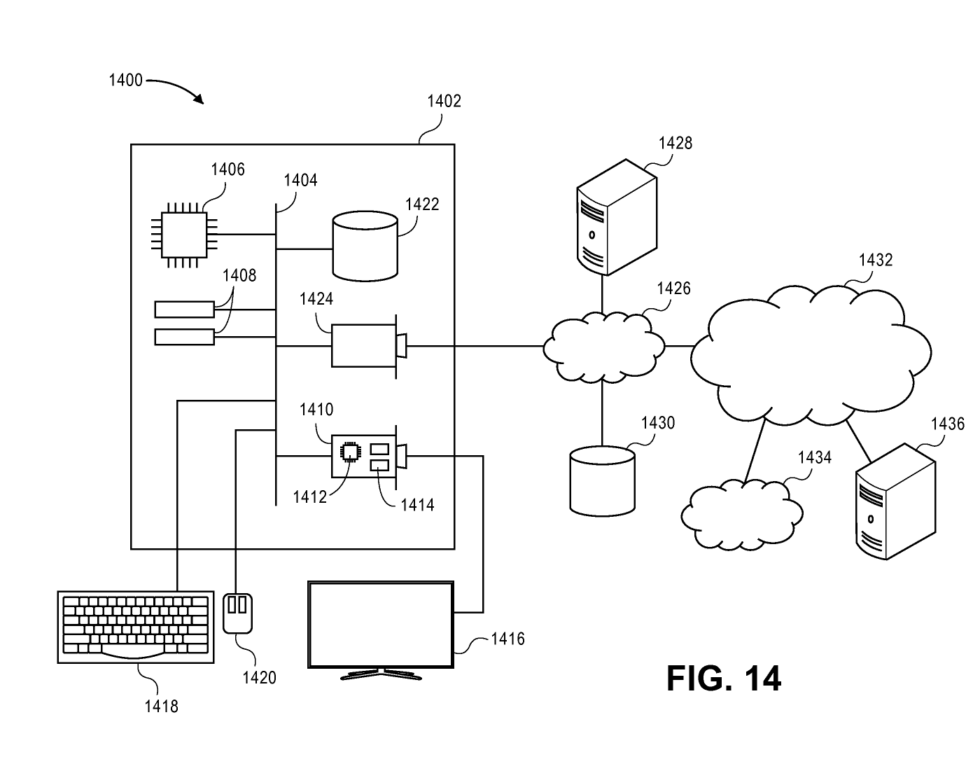

Turning to FIG. 14, the physical hardware that makes up the system will now be discussed. The system 1400 comprising an exemplary hardware platform that can form one element of certain embodiments of the invention is depicted. Computer 1402 can be a desktop computer, a laptop computer, a server computer, a mobile device such as a smartphone or tablet, or any other form factor of general- or special-purpose computing device. Depicted with computer 1402 are several components, for illustrative purposes. In some embodiments, certain components may be arranged differently or absent. Additional components may also be present. Included in computer 1402 is system bus 1404, whereby other components of computer 1402 can communicate with each other. In certain embodiments, there may be multiple busses or components may communicate with each other directly. Connected to system bus 1404 is central processing unit (CPU) 1406. Also attached to system bus 1404 are one or more random-access memory (RAM) modules 1408.

Also attached to system bus 1404 is graphics card 1410. In some embodiments, graphics card 1404 may not be a physically separate card, but rather may be integrated into the motherboard or the CPU 1406. In some embodiments, graphics card 1410 has a separate graphics-processing unit (GPU) 1412, which can be used for graphics processing or for general purpose computing (GPGPU). Also on graphics card 1410 is GPU memory 1414. Connected (directly or indirectly) to graphics card 1410 is display 1416 for user interaction. In some embodiments no display is present, while in others it is integrated into computer 1402. Similarly, peripherals such as keyboard 1418 and mouse 1420 are connected to system bus 1404. Like display 1416, these peripherals may be integrated into computer 1402 or absent. Also connected to system bus 1404 is local storage 1422, which may be any form of computer-readable media, and may be internally installed in computer 1402 or externally and removably attached.

Finally, network interface card (NIC) 1424 is also attached to system bus 1404 and allows computer 1402 to communicate over a network such as network 1426. NIC 1424 can be any form of network interface known in the art, such as Ethernet, ATM, fiber, Bluetooth, or Wi-Fi (i.e., the IEEE 802.11 family of standards). NIC 1424 connects computer 1402 to local network 1426, which may also include one or more other computers, such as computer 1428, and network storage, such as data store 1430. Local network 1426 is in turn connected to Internet 1432, which connects many networks such as local network 1426, remote network 1434 or directly attached computers such as computer 1436. In some embodiments, computer 1402 can itself be directly connected to Internet 1432.

The computer program of embodiments of the invention comprises a plurality of code segments executable by a computing device for performing the steps of various methods of the invention. The steps of the method may be performed in the order described, or they may be performed in a different order, unless otherwise expressly stated. Furthermore, some steps may be performed concurrently as opposed to sequentially. Also, some steps may be optional. The computer program may also execute additional steps not described herein. The computer program, system, and method of embodiments of the invention may be implemented in hardware, software, firmware, or combinations thereof using a user engagement system, which broadly comprises server devices, computing devices, and a communications network.

The computer program of embodiments of the invention may be responsive to user input. As defined herein user input may be received from a variety of computing devices including but not limited to the following: desktops, laptops, calculators, telephones, smartphones, or tablets. The computing devices may receive user input from a variety of sources including but not limited to the following: keyboards, keypads, mice, trackpads, trackballs, pen-input devices, printers, scanners, facsimile, touchscreens, network transmissions, verbal/vocal commands, gestures, button presses or the like.

The server devices and computing devices may include any device, component, or equipment with a processing element and associated memory elements. The processing element may implement operating systems, and may be capable of executing the computer program, which is also generally known as instructions, commands, software code, executables, applications (“apps”), and the like. The processing element may include processors, microprocessors, microcontrollers, field programmable gate arrays, and the like, or combinations thereof. The memory elements may be capable of storing or retaining the computer program and may also store data, typically binary data, including text, databases, graphics, audio, video, combinations thereof, and the like. The memory elements may also be known as a “computer-readable storage medium” and may include random access memory (RAM), read only memory (ROM), flash drive memory, floppy disks, hard disk drives, optical storage media such as compact discs (CDs or CDROMs), digital video disc (DVD), and the like, or combinations thereof. In addition to these memory elements, the server devices may further include file stores comprising a plurality of hard disk drives, network attached storage, or a separate storage network.

The computing devices may specifically include mobile communication devices (including wireless devices), work stations, desktop computers, laptop computers, palmtop computers, tablet computers, portable digital assistants (PDA), smart phones, smart watches, other smart wearables, and the like, or combinations thereof. Various embodiments of the computing device may also include voice communication devices, such as cell phones and/or smart phones. In some embodiments, the computing device will have an electronic display operable to display visual graphics, images, text, etc. In certain embodiments, the computer program facilitates interaction and communication through a graphical user interface (GUI) that is displayed via the electronic display. The GUI enables the user to interact with the electronic display by touching or pointing at display areas to provide information to the system.

The communications network may be wired or wireless and may include servers, routers, switches, wireless receivers and transmitters, and the like, as well as electrically conductive cables or optical cables. The communications network may also include local, metro, or wide area networks, as well as the Internet, or other cloud networks. Furthermore, the communications network may include cellular or mobile phone networks, as well as landline phone networks, public switched telephone networks, fiber optic networks, or the like.

The computer program may run on computing devices or, alternatively, may run on one or more server devices. In certain embodiments of the invention, the computer program may be embodied in a stand-alone computer program (i.e., an “app”) downloaded on a user's computing device or in a web-accessible program that is accessible by the user's computing device via the communications network. As used herein, the stand-along computer program or web-accessible program provides users with access to an electronic resource from which the users can interact with various embodiments of the invention.

In embodiments of the invention users may be provided with different types of accounts. Each type of user account may provide their respective users with unique roles, capabilities, and permissions with respect to implementing embodiments of the invention. For instance, a caregiver may be provided with a caregiver account that permits the caregiver to access embodiments of the invention that are applicable to the caregiver managing the treatment of multiple patients. Additionally, a patient may be provided with a patient account that permits the patient to access embodiments of the invention that are applicable to managing the their own treatment. A friends or family member of the patient may be provided with a helper account to access embodiments of the invention that are applicable to assisting the patient in the patient's treatment. In addition, any number and/or any specific types of accounts is provided as may be necessary to carry out the functions, features, and/or implementations of the invention. Upon a caregiver, patient, or helper logging in to the electronic resource for a first time, the caregiver, patient, or helper may be required to provide various items of identification information to create their respective accounts. Such identification information may include, for instance, personal name, business name, email address, phone number, or the like. Upon providing the identification information, the user may be required to enter (or may be given) a username and password, which will be required to access the electronic resource.

Execution of the computer program of embodiments of the invention performs steps of the method of embodiments of the invention. Because multiple users may be updating information stored, displayed, and acted upon by the computer program, information displayed by the computer program is displayed in real-time. “Real-time” as defined herein is when the processing element of the system 10 performs the steps less than every 1 second, every 500 milliseconds, every 100 milliseconds, or every 16 milliseconds.

The method of embodiments of the invention for providing the GUI broadly comprises the following steps: accepting user input, updating information in response to user input, providing an updated GUI. Initialization of the computer program by a user, as may occur when a new user begins to use the system, includes the following additional steps: prompting for the user login, retrieving the credentials input by the user, and determining the first screen of the GUI to provide to the user. Additional steps are also performed if the user has never used the system before, namely prompting the user to create a login and verifying the login is available.

The method of embodiments of the invention for providing the secondary GUI broadly comprises the following steps: accepting user input, updating information in response to user input, and providing an updated GUI. Each time the user opens the computer program comprises the following additional steps: verifying the credentials of the user, determining the first screen of the GUI to provide to the user. The secondary GUI is deployed in response to the message sent to the user so creating a login does not need to be performed.

Although embodiments of the invention have been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A user engagement system for providing a regimen to a user, the system comprising:
a predictive engine for analyzing a set of user information,
said predictive engine analyzing an attribute pool to determine a set of recommended attributes,
wherein the attribute pool includes a plurality of possible attributes that can be adapted into the regimen,
wherein the set of recommended attributes are selected based upon said analysis of the set of user information;
an implementation engine for displaying the regimen to the user,
said implementation engine monitoring an adherence level of the user; and
a responsive engine for adapting the regimen to improve the adherence level upon the patient falling below a threshold level,
said responsive engine determining a set of revised attributes that are likely to improve the adherence level of the user.

2. The system of claim 1, wherein the set of user information includes at least one of demographic information related to the user, a geographic location of the user, and an education level of the user.

3. The system of claim 1,
wherein the user is a patient,
wherein the regimen is a medical regimen,
wherein the medical regimen relates to a medical diagnosis.

4. The system of claim 3, wherein the set of user information includes a set of caregiver recommendations and at least a portion of a medical history for the user.

5. The system of claim 1, wherein the set of user information includes a set of device information related to a device associated with the user.

6. The system of claim 1, wherein the attribute pool includes a plurality of verbiage levels,
wherein the verbiage level is selected, based upon the analysis of user information, to appeal to the user.

7. The system of claim 1, wherein the attribute pool includes a plurality of subject contents,
wherein each subject content includes information about an aspect of the regimen.

8. The system of claim 1, wherein the attribute pool includes a plurality of interfaces, a plurality of games, and a plurality of reminder schedules.

9. A computerized method for providing a regimen to a user, the method comprising the following steps:
receiving, from a predictive engine, a set of recommended attributes for the regimen;
creating the regimen based upon the set of recommended attributes;
presenting an educational component of the regimen to the user;
presenting a reminder component of the regimen to the user;
monitoring activities of the user to determine an adherence level of the user;
initiating, upon the adherence level falling below a threshold, communication between the user and a third party;
analyzing the communication between the user and the third party to determine a reason for non-adherence;
sending, to a responsive engine, a set of adherence information indicative of the activities of the user; and
receiving, from the responsive engine, a set of revised attributes for the regimen.

10. The computerized method of claim 9, further comprising the following steps:
presenting a game component of the regimen to the user; and
presenting an incentive component of the regimen to the user.

11. The computerized method of claim 9, wherein said communication between the user and the third party is analyzed to determine an effectiveness of the communication.

12. The computerized method of claim 9,
wherein the user is a patient,
wherein the regimen is a medical regimen associated with a medical diagnosis,
wherein the third party is a caregiver of the user.

13. The computerized method of claim 9, further comprising the following step:
creating a new regimen based upon the set of revised attributes.

14. A system for improving adherence by a user to a regimen, the system comprising:
a predictive engine for determining a set of recommended attributes for the regimen;
an implementation engine for displaying the regimen to the user,
said implementation engine monitoring an adherence level of the user;
a responsive engine for determining a set of revised attributes designed to improve the adherence level of the user,
wherein the revised attributes are based at least in part on an analysis of a non-adherence pattern displayed by the user,
wherein the revised attributes are based at least in part on a comprehension level of the user,
said responsive engine accessing an attribute pool to determine attributes that are likely to improve the adherence level of the user.

15. The system of claim 14, wherein the responsive engine is triggered by receiving information indicative that the adherence level of the user has dropped below a threshold.

16. The system of claim 14, wherein the set of revised attributes includes attributes from the attribute pool that were not in the set of recommended attributes.

17. The system of claim 14, wherein the set of revised attributes includes at least one attribute that has been modified from the set of recommended attributes.

18. The system of claim 14, wherein the responsive engine analyzes changes in a set of user information since the predictive engine determined the set of recommended attributes.

19. The system of claim 14, wherein the responsive engine analyzes prior sets of revised attributes in determining the set of revised attributes.

20. The system of claim 14, wherein the responsive engine sends the set of revised attributes to the implementation engine, such that the implementation engine presents the regimen to the user based upon the set of revised attributes.
